(19) United States
(12) Patent Application Publication
(10) Pub. No.: US 2021/0121606 A1
(43) Pub. Date: Apr. 29, 2021

(54) IONIC-DOPED COMPOSITION METHODS AND USES THEREOF

(71) Applicant: ASSOCIATION FOR THE ADVANCEMENT OF TISSUE ENGINEERING CELL BASED TECHNOLOGIES & THEAPIES (A4TEC), Braga (PT)

(72) Inventors: Sandra Cristina DE ALMEIDA PINA, Vila Do Conde (PT); Viviana PINTO RIBEIRO, Vila Pouca De Aguiar (PT); Joaquim Miguel ANTUNES DE OLIVEIRA, Braga (PT); Rui Luís GONÇALVES DOS REIS, Porto (PT)

(21) Appl. No.: 16/617,415

(22) PCT Filed: May 28, 2018
(86) PCT No.: PCT/IB2018/053783

§ 371 Date: Nov. 26, 2019

(30) Foreign Application Priority Data
May 26, 2017 (PT) 110106

(51) Int. Cl. A61L 27/46; A61L 27/54; A61L 27/56

(52) U.S. Cl. CPC A61L 27/46; A61L 27/54; A61L 2400/12; A61L 2430/02; A61L 2400/06; A61L 27/56

(57) ABSTRACT

The present disclosure concerns the production of an ionic-doped composition and nanocomposites hierarchically structured incorporating bioactive ions, and its use in regenerative medicine and/or tissue engineering.

### TECHNICAL DOMAIN

The present disclosure concerns the production ionic-doped composition and nanocomposites hierarchically structured incorporating bioactive ions, and its use in regenerative medicine and/or tissue engineering.

### BACKGROUND ART

Bone defects are often associated to a disease state (e.g. Osteoarthritis (OA), Osteoporose (OP), osteomyelitis, and osteogenesis imperfect) and trauma related injuries resulting from primary tumor resection and orthopaedic surgeries (e.g. total joint arthroplasty and implant fixation). In addition, spinal fractures, called vertebral compression fractures, are the most common fracture in patients with OP, affecting nearly 700,000 people each year, typically postmenopausal women. However, others fractures like fractures of the hip, wrist, and proximal humerus are commonly observed in patients with OP. Novel biomaterials that can provide temporary structural support to the damaged region, initiate the cascade of osteogenesis and mineralized matrix formation, and degrade concurrent with the production of ECM, are urgently needed for limb, head, and face reconstruction of patients with multiple traumatic injuries. Nevertheless, concern issues are associated with risk of disease transfer, infection, chronic pain, possible immunogenicity, deficient supply, and increase operative time and cost. Bioactive calcium phosphate (CaP) ceramics have been used in orthopaedics and maxillofacial surgery but, due to low initial strength, their use is limited to defects that are subject to uniform loading. Composites made of CaP nanopowders and different biopolymers have been developed for bone TE scaffolding, mainly due to the enhanced mechanical properties of the final materials as compared with their single-phase constituents. The materials with nanosized features have large surface area offering improved mechanical properties, while maintaining the favourable osteoconductivity and biocompatibility of the materials.

The presence of different ions in the nanocomposites is a way to improve biofunctioning and tissue regeneration by means of not only stimulating and tuning host healing response at the site of injury to facilitate the tissue repair (e.g. osteogenesis and vascularization), but also to mimic native tissue organization with the ultimate goal of achieving a fully integrated and functional engineered tissue. The incorporation of Sr, Zn, and Mn, Mg and Ga present beneficial effects on bone regeneration, and it increases endothelial cells proliferation and tubule formation, controlled degradation, as well as the mechanical strength of the nanomaterials.

Silk fibroin from the silk worm Bombyx mori, has often been used as a textile material, yet, more and more attention has been given to silk lately due to its appropriate processing, biodegradability and the presence of easy accessible chemical groups for functional modifications. Studies suggest that silk is not only biodegradable but also bioresorbable, characteristics for tissue engineering and regenerative medicine. The major advantage of silk compared to other natural biopolymers is its excellent mechanical property. Other important advantages include good biocompatibility, water-based processing, biodegradability and the presence of easy accessible chemical groups for functional modifications.

Aqueous solutions of silk fibroin with different concentrations work as precursors for the formation of the hydrogels. The silk fibroin solutions are surprisingly capable for forming hydrogels in the presence of horseradish peroxidase and hydrogen peroxide (oxidizer) at mild temperatures within physiological pH.

Document CN 200710069129 described a method for the preparation process of silk fibroin/calcium carbonate nanocomposites. It is prepared through one biological mineralizing process simulating that in shell growth.

These facts are disclosed in order to illustrate the technical problem addressed by the present disclosure.

### GENERAL DESCRIPTION

The present disclosure concerns the production ionic-doped composition and nanocomposites hierarchically structured incorporating bioactive ions, and its use in regenerative medicine and/or tissue engineering.

The present disclosure also relates to method for producing hierarchical nanocomposites structures of enzymatically cross-linked silk fibroin hydrogels and calcium phosphates nanopowders (e.g., β-tricalcium phosphate and α-tricalcium phosphate, hydroxyapatite) doped with different ions (e.g. Zn, Sr, Mn, Mg, and Ga).

Silk fibroin contains around 5 mol % tyrosine groups, which are oxidized by peroxidase/hydrogen peroxide and subsequently cross-linked to form a three-dimensional network. Silk fibroin hydrogels are achieved by the cross-linking of tyrosine groups in silk fibroin. This cross-link surprisingly leads to a stronger and more stable three-dimensional network, thus conferring the scaffold higher mechanical properties, more elasticity and a lower degradation rate, when compared to tubes that did not undergo this cross-link before turning in β-sheet conformation.

An aspect of the present disclosure relates to a composition for use in regenerative medicine and/or tissue engineering comprising:
an enzymatically crosslinked silk fibroin and, a plurality of ionic doped calcium phosphate nanoparticles.

Another aspect of the present disclosure relates to a composition for use in regenerative medicine and/or tissue engineering comprising enzymatically crosslinked silk fibroin and ionic doped calcium phosphate nanoparticles, wherein said composition is administrated in composite comprising
an enzymatically crosslinked silk fibroin and, a plurality of ionic doped calcium phosphate nanoparticles. The obtained composite is monolithic and hierarchically structured.

In an embodiment, the composition may comprise 10-20% (w/w) of an enzymatically crosslinked silk fibroin, preferably 12-16% (w/w, more preferably 15-16% (w/w).

In an embodiment, the calcium phosphates nanoparticles may be selected from a list consisting of: α or β-tricalcium phosphate, hydroxyapatite, calcium peroxide or other oxidizer, or mixtures thereof.

In an embodiment, the nanoparticle size may be between 1-100 nm, preferably 10-50 nm, more preferably 20-30 nm.

In an embodiment, crosslinked of silk fibroin may be obtainable by an enzymatic reaction with horseradish peroxidase and hydrogen peroxide.

In an embodiment, the ion may be selected from a list consisting of: strontium, zinc, manganese, silicon, magnesium, gallium, lithium, or mixtures thereof.

In an embodiment, the ionic-doped nanoparticles contents may be up to 20 wt. %, preferably between 10-18 wt. %, more preferably 16-20 wt. %.

In an embodiment, the ionic-doped nanoparticles may content up to 10 mol. % of ionic dopants, preferably between 5-10 mol. %, more preferably 8-10 mol. %.

In an embodiment, the composition may further comprise a bioactive molecule and/or an active ingredient.

In an embodiment, the bioactive molecule/active ingredient is selected from the group consisting of: growth factors, hemostatic agents, osteoconductive agents, antibiotics, anti-inflammatory agents, anti-cancer agents, cells, an antiseptic agent, an antipyretic agent, an anaesthetic agent, a therapeutic agent, or mixtures thereof.

In an embodiment, the composition may be use for the repair, treatment or regeneration of bone, or cartilage or osteochondral, namely fractures or defects.

In an embodiment, the composition may be administrated as an injectable form.

Another aspect of the present invention is related to a scaffold or composite comprising the composition described in the present disclosure comprising a porosity between 40-80%, a pore size between 150-350 μm, in particular 200-300 μm.

The porosity may be measured by several methods, in the present disclosure the porosity was measured through 3D microcomputed tomography morphometric analysis.

Another aspect of the present invention is related to a prosthesis coated with the composition described in the present subject-matter.

In an embodiment, compounds made of inorganic calcium phosphates (CaP) (e.g: β- and α-TCP, HAp) are frequently used because they have remarkable biocompatibility and osteoconductivity, and do not cause cell death in the surrounding tissues. The biological response to these materials follows a similar cascade observed in fracture healing. CaP can undergo processes of dissolution and precipitation resulting in a strong material-bone interface. Although β-TCP and HAp have similarities in their chemical composition, they differ in their biological resorbing capability. The resorption of a ceramic HAp is slow, and once implanted into the body, HAp may remain integrated into the regenerated bone tissue, while β-TCP is completely reabsorbed. Clinical applications of pure HAp can be improved with the bioresorbable β-TCP for better bone regeneration.

In an embodiment, the materials with nanosized features can intensely change the physical properties of the polymer matrix.

In an embodiment, the nanopowders have large surface area when compared to the conventional micronized materials, which can form a tight interface with the polymeric matrices, offering improved mechanical properties, while maintaining the favourable osteoconductivity and biocompatibility of the materials, thus influencing protein adsorption, cells adhesion, proliferation and differentiation for new tissue formation.

In an embodiment, the ionic incorporation into the structure of CaP, namely in β-TCP and Hap, can affect the lattice structure, microstructure, crystallinity, dissolution rate, and biological processes of CaPs.

In an embodiment, the ionic-doped CaP nanopowders are obtained via aqueous precipitation from precursors of Ca, P, and precursors nitrates of ionic dopants, in a medium of controlled pH, followed by heat treatment.

In an embodiment, CaP nanopowders may be determined by XRD and FTIR techniques, to assess their crystallinity and the presence of functional groups.

In an embodiment, the incorporation of ionic doping elements into the CaP nanopowders may be calculated on the basis of XRD patterns through Rietveld analysis.

In an embodiment, enzymatically cross-linked SF hydrogels/ionic-doped CaP nanocomposites are prepared using the following procedure:
preparing an aqueous silk fibroin (SF) solution with a final concentration of 16-20 wt. %;
adding of horseradish peroxidase (50 μL/ml of silk solution) and hydrogen peroxide (65 μL/ml of silk solution);
adding of ionic-doped CaP nanopowders with 0-10 mol. %;
adding NaCl particles (sizes of 250-500 μm);
incubating the whole system at 37° C. for 2 hours for the complete formation of the hydrogel;
salt-leaching for 1 day of the scaffolds;
freeze-drying of the scaffolds up to 4 days.

In an embodiment, the microstructure of the scaffolds may be determined by Micro-CT 3D reconstructions in which morphometric parameters such as total % of porosity, mean pore size and trabecular thickness will be quantified.

In an embodiment, the mechanical properties of the scaffolds may be determined by DMA and compressive strength in dry and wet state.

In an embodiment, the presence of different ions in the nanocomposites is a way to improve tissue biofunctioning and regeneration by means of stimulating and tuning host healing response at the site of injury to facilitate the tissue repair (e.g. osteogenesis and vascularization). The incorporation of Sr, Zn, and Mn, Mg and Ga present beneficial effects on bone regeneration, and it increase endothelial cells proliferation and tubule formation, and controlled degradation of the nanomaterials.

### DESCRIPTION OF THE DRAWINGS

The following figures provide preferred embodiments for illustrating the description and should not be seen as limiting the scope of disclosure.

FIG. 1—XRD patterns of SF/pure TCP and SF/ZnSrTCP nanocomposites.

FIG. 2—μ-CT images of the scaffolds: A) 3D acquisition, B) morphometric analysis, and C) 2D porosity of bone and cartilage parts.

FIG. 3—SEM/EDS analyses of: A) SF/ZnSrTCP nanocomposites, showing the different SF, interface and SF/ZnSrTCP layers, and B) respective EDS elemental analysis of SF layer (left) and SF/ZnSrTCP layer (right).

FIG. 4—DMA analysis of the scaffolds.

FIG. 5—Compressive strength A) and compressive modulus B) of the scaffolds.

FIG. 6—Histological and immunofluorescence analysis of the hOBs and hACs co-cultured in the BdTCP scaffolds for 1, 7 and 14 days. Standard H&E staining was used to evaluate cell distribution and ECM formation. Sirius red (red) staining was used for the visualization of collagen at the ECM, Safranin-O (red) staining was used to detect GAGs formation (scale bar: 200 μm). Representative immunofluorescence images of the osteogenic-related marker OPN (green) and chondrogenic-related marker ACAN (red) in the co-culture system. Nuclei are stained in blue (scale bar: 100 μm). The red arrow indicates a stained area of ECM mineralization. The yellow arrows indicate the stained SF and SF-dTCP layers.

FIG. 7—Histological and immunofluorescence analysis of the hOBs and hACs co-cultured in the BTCP scaffolds for 1, 7 and 14 days. Standard H&E staining was used to evaluate cell distribution and ECM formation. Sirius red (red) staining was used for the visualization of collagen at the ECM, Safranin-O (red) staining was used to detect GAGs formation (scale bar: 200 μm). Representative immunofluorescence images of the osteogenic-related marker OPN (green) and chondrogenic-related marker ACAN (red) in the co-culture system. Nuclei are stained in blue (scale bar: 100 μm). The red arrow indicates a stained area of ECM mineralization. The yellow arrows indicate the stained SF and SF-TCP layers.

### DETAILED DESCRIPTION

The present disclosure concerns the production ionic-doped composition and nanocomposites hierarchically structured incorporating bioactive ions, and its use in regenerative medicine and/or tissue engineering.

The present disclosure present disclosure also relates to method for producing hierarchical nanocomposites structures of enzymatically cross-linked silk fibroin hydrogels and calcium phosphates nanopowders (e.g., β- and α-tricalcium phosphate, hydroxyapatite) doped with different ions (e.g. Zn, Sr, Mn, Mg, and Ga).

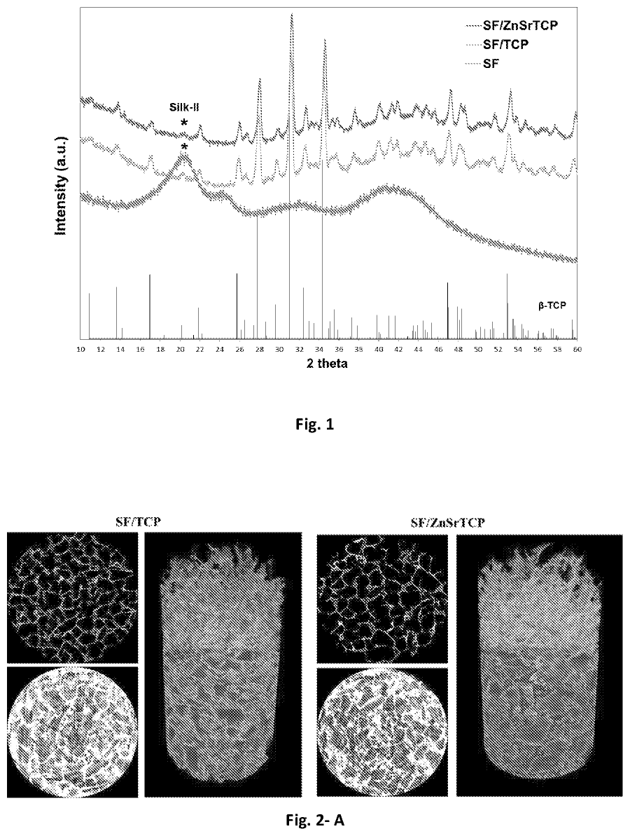

In FIG. 1 can be observed that the scaffolds show low intensity peaks located at 20.2° corresponding to the β-sheet crystalline structure (silk-II structure) of native silk fibroin, and the characteristic phases of β-TCP and β-calcium pyrophosphate (CPP) belonging to the TCP and ZnSrTCP powders. These results indicate that the powders were successfully incorporated into the SF, and this fact did not change its structure of β-sheet conformation, which is critical for the maintenance of the mechanical properties and structural stability of the scaffolds.

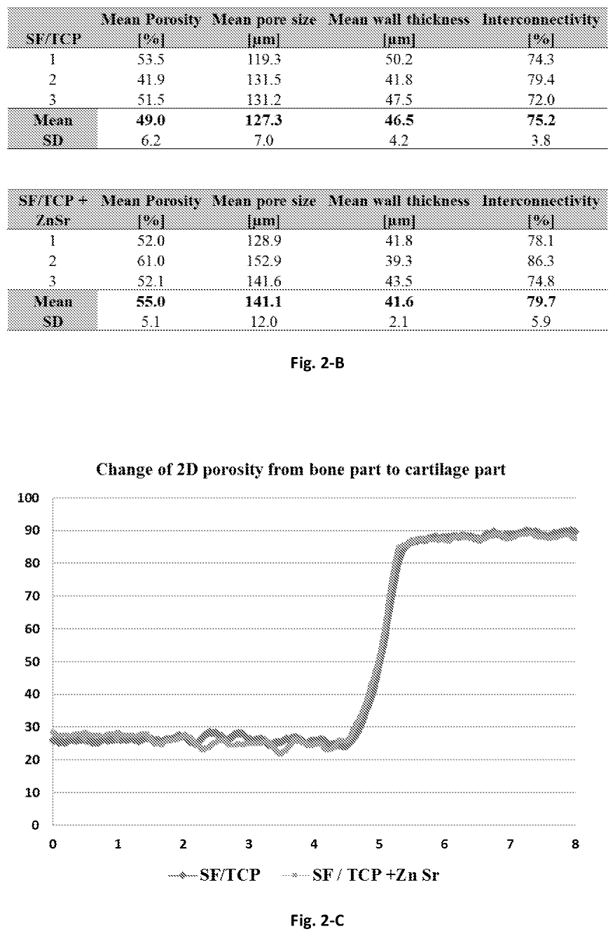

In FIG. 2A is possible to observe the porous structure in each layer of the scaffolds with the TCP powder retained only in the composite layers, as confirmed by the blue domain present in the 3D reconstructions scaffolds. In FIG. 2C can be observed that the porosity distribution profile is homogeneous in each scaffold layer; however, a substantial increase of porosity is observed from the interface region until the silk fibroin layers.

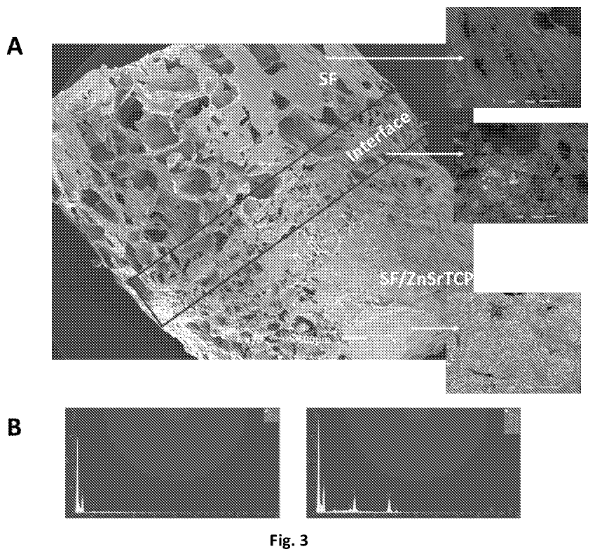

In FIG. 3A can be observed that the scaffolds presented a macro- and micro-porous structure on both layers, presenting macro-pores larger than 500 μm and micro-pores that reach 10 μm. The scaffold layers were well integrated by continuous interface regions of ˜500 μm thickness. From FIG. 3B is possible to see calcium (Ca) and phosphorous (P) ions in the subchondral bone-like layers and interface regions, as well as the presence of Zn and Sr peaks.

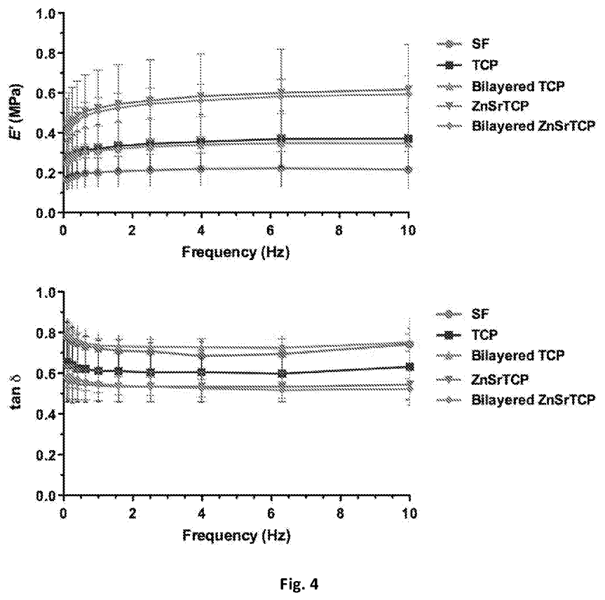

In FIG. 4 is observed that the storage modulus (E′) of the bilayered and monolayered scaffolds increased at lower rates, with increasing testing frequencies (from 0.1 to 10 Hz), ranging from 0.40±0.11 to 0.59±0.21 MPa on bilayered ZnSrTCP scaffolds, 0.26±0.06 to 0.35±0.09 MPa on bilayered TCP scaffolds, and 0.18±0.05 to 0.24±0.09 MPa on SF scaffolds. The loss factor (tan δ) obtained for the bilayered and monolayered control scaffolds were constant when the frequency increased from 0.1 to 10 Hz. All groups of scaffolds presented similar and high loss factor values for the tested frequencies.

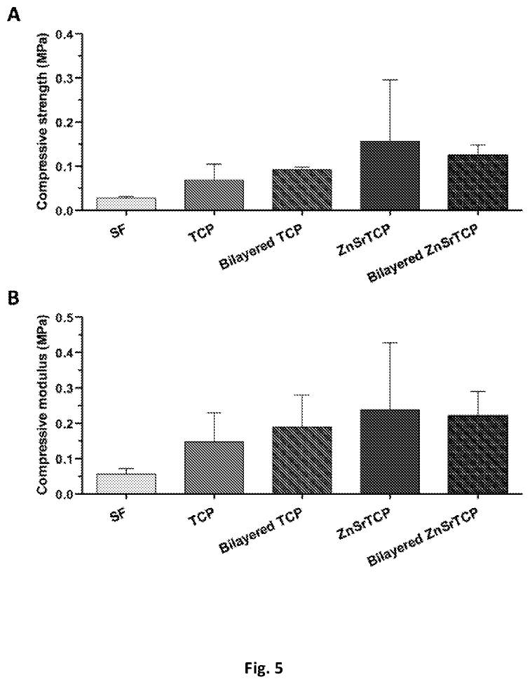

In FIG. 5A and B, the wet compressive modulus of the bilayered ZnSrTCP (0.23±0.06 MPa) and bilayered TCP (0.19±0.09 MPa) scaffolds was higher than that obtained for the corresponding monolayered scaffolds (SF: 0.06±0.04 MPa; SF/ZnSrTCP: 0.17±0.11 MPa; SF/TCP: 0.15±0.08 MPa). The SF scaffolds presented the lowest compressive modulus, as compared to the bilayered (B) and monolayered composite scaffolds.

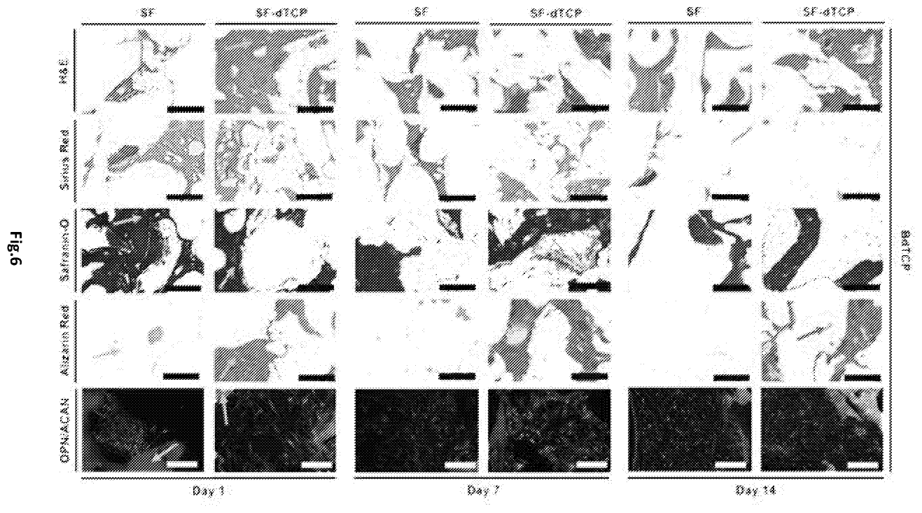

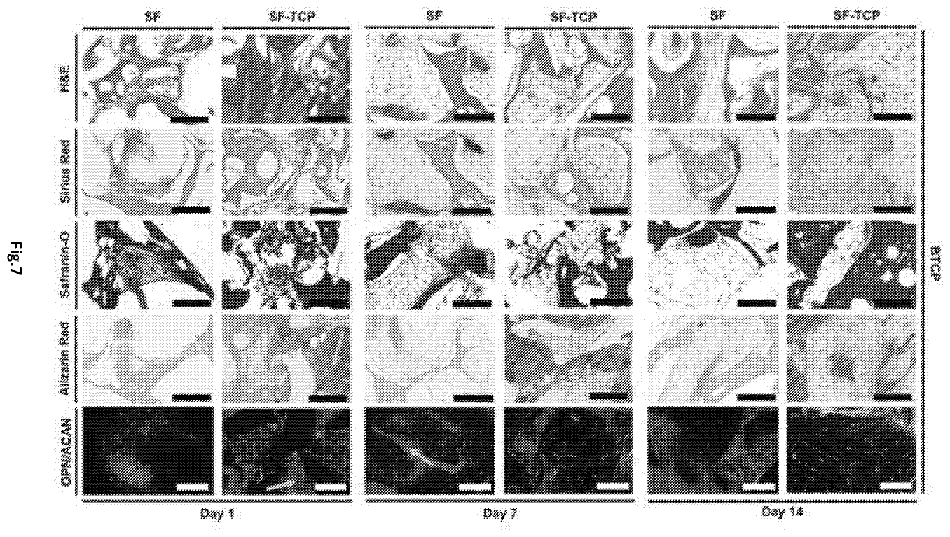

In FIGS. 6 and 7, histological and immunofluorescence analysis was performed to assess the phenotypic expression and activity of the osteoblasts and chondrocytes co-cultured in the BdTCP (FIG. 6, wherein B is bilayered and d is ZnSr) and BTCP (FIG. 7) constructs. The results obtained from the H&E staining images, showed that the hOBs and hACs were able to proliferate up to 14 days of culture. At day 7 and day 14, the hACs attached to the macro-pores walls of the SF layers formed self-aggregated clusters, whereas the hOBs spread and filled the inner porous of the SF-dTCP and SF-TCP layers. The newly formed ECM was stained with Sirius red, showing after 14 days of culture a well pronounced collagen matrix deposited in the co-cultured BdTCP and BTCP scaffolds. In the SF layers, the collagen matrix was mainly evidenced in the hACs aggregates. The GAGs deposition on the BdTCP and BTCP constructs was observed at day 14, by the positive staining for safranin-O. An increase of the ECM mineralization was observed up to 14 days of culture in the SF-dTCP and SF-TCP layers, as compared to the lower staining intensity observed on the SF layers. Since the hACs tend to form thick self-aggregated clusters, the staining intensity in these clusters was considerably higher. Up to 14 days of culture, no detectable differences were observed in the type of ECM produced by the hOBs and hACs co-cultured in the BdTCP and BTCP constructs, with that produced on the corresponding monolayered control scaffolds.

### Preparation of Silk Fibroin

In an embodiment, the silk fibroin (SF) is extracted from Bombyx mori cocoons, by removing the sericin with boiling the cocoons in a 0.02 M Na₂CO₃ solution for 1 h, and then rinsing with distilled water. The resulting SF are dissolved in 9.3 M LiBr at 70° C. for 1 h, and then dialyzed against distilled water by using a benzoylated dialysis tubing for 48 h. Afterwards, the SF solution are concentrated by dialysis in a 20 wt. % of PEG solution for 6 h, to yield a solution of 16 wt. %. The tubing will be rinsed in distilled water, and the solution will be collected.

### Ionic-Doped Calcium Phosphates Preparation

In an embodiment, the ionic-doped nanopowders are obtained by aqueous precipitation from calcium nitrate tetrahydrate (Ca(NO₃)4H₂O) and diammonium hydrogen phosphate ((NH₄)₂HPO₄) in a medium of controlled pH with the addition of NH₄OH. Ionic-doped nanopowders (0-10 mol. %) are synthesized by adding suitable amounts of the precursor nitrates of the doping elements. The precipitated suspensions are kept for 4 h under constant stirring conditions and matured for further 20 h under rest conditions, at 20-50° C. The resulting precipitates are vacuum filtered, dried at 100° C., and heat treated for 2 h at 1000-1100° C. The nanopowders are grounded under dry conditions in a planetary mill, followed by sieving.

### Fabrication of Monolithic and Hierarchically Structured Nanocomposites

In an embodiment, the SF hydrogels are prepared by using SF solution of 16 wt. % concentration, horseradish peroxidase solution (HRP, 50 μL/mL of SF) and hydrogen peroxide (65 μL/ml of SF). The nanocomposites are obtained by mixing SF solution with varied amount of HRP and H₂O₂ solutions, followed by addition of ionic-doped CaP nanopowders (16-20 wt. %, CaP mass divided by the total mass of SF) and NaCl particles. The gelation process is performed at 37° C. The salt is extracted by immersion in distilled water for 1 day. The nanocomposites are frozen at −80° C. followed by lyophilization up to 4 days.

All references recited in this document are incorporated herein in their entirety by reference, as if as each and every reference had been incorporated by reference individually.

Those skilled in the art will recognize or be able to ascertain using no more than routine experimentation, many equivalents to the specific embodiments of the invention described herein. The scope of the present invention is not intended to be limited to the above description, but rather is as set forth in the appended claims.

Where singular forms of elements or features are used in the specification of the claims, the plural form is also included, and vice versa, if not specifically excluded. For example, the term “a cell” or “the cell” also includes the plural forms “cells”. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

The aforementioned embodiments are combinable.

The following claims further set out particular embodiments of the disclosure.

The following references should be considered herewith incorporated in their entirety:
1. Yan L. P., Silva-Correia J., Ribeiro V. P., Miranda-Gonçalves V., Correia C., da Silva Morals A., Sousa R. A., Reis R. M., Oliveira A. L., Oliveira J. M., and Reis R. L., “, Scientific Reports, vol. 6, issue 31037, doi:10.1038/srep31037, 2016.
2. Yan L. P., Salgado A. J., Oliveira J. M., Oliveira A. L., and Reis R. L., “De novo bone formation on macro/microporous silk and silk/nano-sized calcium phosphate scaffolds”, Journal of Bioactive and Compatible Polymers, vol. 28, issue 5, pp. 439-452, doi:0.1177/0883911513503538, 2013.
3. Yan L. P., Silva-Correia J., Correia C., Caridade S. G., Fernandes E. M., Sousa R. A., Mano J. F., Oliveira J. M., Oliveira A. L., and Reis R. L., “Bioactive Macro/micro Porous Silk Fibroin/Nano-sized Calcium Phosphate Scaffolds with potential for Bone Tissue Engineering Applications”, Nanomedicine, Nanomedicine, vol. 8, issue 3, pp. 359-378, doi:10.2217/nnm.12.118, 2012.
4. Yan L. P., Silva-Correia J., Oliveira M. B., Vilela C. A., Pereira H., Sousa R. A., Mano J. F., Oliveira A. L., Oliveira J. M., and Reis R. L., “Bilayered Silk/Silk-NanoCaP Scaffolds for Osteochondral Tissue Engineering: In Vitro and in Vivo Assessment of Biological Performance”, Acta Biomaterialia, vol. 12, issue 2015, pp. 227-241, doi:10.1016/j.actbio.2014.10.021, 2014.
5. Yan L. P., Oliveira J. M., Oliveira A. L., Caridade S. G., Mano J. F., and Reis R. L., “Macro/micro Porous Silk Fibroin Scaffolds with Potential for Articular Cartilage and Meniscus Tissue Engineering Applications”, Acta Biomaterialia, vol. 8, issue 1, pp. 289-301, 2012.
6. VAN T. D., TRAN N. Q., NGUYEN D. H., NGUYEN C. K., TRAN D. L., NGUYEN P. T. “Injectable Hydrogel Composite Based Gelatin-PEG and Biphasic Calcium Phosphate Nanoparticles for Bone Regeneration”, Journal of ELECTRONIC MATERIALS, Vol. 45, No. 5, 2016, DOI: 10.1007/s11664-016-4354-3.

1. A composition for use in regenerative medicine and/or tissue engineering comprising:
an enzymatically crosslinked silk fibroin; and
a plurality of ionic doped calcium phosphate nanoparticles.

2. (canceled)

3. The composition according to claim 1, comprising 10-20% (w/w) of the enzymatically crosslinked silk fibroin.

4. The composition according to claim 1, wherein the ionic doped calcium phosphate nanoparticles are selected from the group consisting of: α-tricalcium phosphate, β-tricalcium phosphate, hydroxyapatite, and mixtures thereof.

5. The composition according to claim 1, wherein the ionic doped calcium phosphate nanoparticle size is between 1-100 nm.

6. The composition according to claim 1, wherein crosslinked of silk fibroin is obtainable by an enzymatic reaction with horseradish peroxidase and hydrogen peroxide, calcium peroxide or an oxidizer.

7. The composition according to claim 1, wherein the ion of the ionic doped calcium phosphate nanoparticle is selected from the group consisting of: strontium, zinc, manganese, silicon, magnesium, lithium, gallium, and mixtures thereof.

8. The composition according to claim 1, comprising ionic-doped nanoparticles up to 20 wt. %.

9. The composition according to claim 1, wherein the ionic-doped nanoparticles contents up to 10 mol. % of ionic dopants.

10. The composition according to claim 1, further comprising a bioactive molecule, an active ingredient, or both the bioactive molecule and the active ingredient.

11. The composition according to claim 1, wherein the bioactive molecule/active ingredient is selected from the group consisting of: a growth factor, a hemostatic agent, an osteoconductive agent, an antibiotic, an anti-inflammatory agent, an anti-cancer agent, cells, an antiseptic agent, an antipyretic agent, an anaesthetic agent, a therapeutic agent, and mixtures thereof.

12. (canceled)

13. (canceled)

14. A scaffold comprising the composition of claim 1, wherein the composition has a porosity between 40-80%, and a pore size between 150-350 μm.

15. (canceled)

16. A method for administering a composite adapted for regenerative medicinal use, tissue engineering, or both, comprising the step of:
administrating in an injectable form an enzymatically crosslinked silk fibroin and a plurality of ionic doped calcium phosphate nanoparticles combined as the composite.
